(19) United States
(12) Patent Application Publication
(10) Pub. No.: US 2010/0022439 A1
(43) Pub. Date: Jan. 28, 2010

(54) Nanostructures with High Load of Active Agents

(75) Inventor: Paul R. Blake, Newtown, PA (US)

Correspondence Address: PATENT DOCKET ADMINISTRATOR;LOWENSTEIN SANDLER PC, 65 LIVINGSTON AVENUE, ROSELAND, NJ, 07068, US

(73) Assignee: PURDUE PHARMA L.P., Stamford, CT (US)

(21) Appl. No.: 12/484,848

(22) Filed: Jun. 15, 2009

Related U.S. Application Data
(60) Provisional application No. 61/061,802, filed on Jun. 16, 2008.

(51) Int. Cl. A61K 38/02; A61K 47/04; B82B 1/00; B82B 3/00

(52) U.S. Cl. 514/2; 514/770; 977/742; 977/906

(57) ABSTRACT

The present invention provides an improved method of loading silica nanostructures with an active agent, comprising exposing a mixture comprising a liquid medium containing an active agent and silica nanostructures to reduced air pressure for a time period, and then returning the mixture to atmospheric air pressure at the end of the time period.

This application claims the benefit of U.S. Provisional Application Ser. No. 61/061,802 filed Jun. 16, 2008, the disclosure of which is incorporated herein bad reference.

### BACKGROUND

One of the new promising trends in drug delivery technology is the use of nanostructures. Nanostructures are typically engineered to be less than 500 nanometers in size and offer greater uptake efficiency by cells due to their tiny dimensions. Because of their size, nanostructures can also be targeted to a specific site, which may be useful in gene therapy and cancer diagnosis and treatment. See e.g., Suri et al., J. Occupational Med. Tox. 2007, 2:16; Cuenca et al. Cancer 2006, 107:3.

Drug delivers, systems utilizing nanostructures have considerable potential for the treatment of many disease states and conditions. The potential advantages of utilizing nanostructures in drug delivers may include increased bioavailability, decreased toxicity and increased stability.

Nanostructures allow for a broader application of potential active agents that can be formulated for therapeutic use and a broader array of potential routes of administration for a given therapeutic agent. For example, an agent that may not have had an adequate bioavailability to be therapeutically useful may potentially be utilized in a bioavailable oral, parenteral or implantable dosage form when incorporated into nanostructures.

The incorporation of active agents into nanostructures can be particularly advantageous in the formulation of controlled release dosage forms, resulting in improved bioavailability and reduced dosing frequency. This could potentially resolve the problem of noncompliance to prescribed therapy, which is one of the major obstacles in the control of many disease states and conditions.

A particular material that has potential for utilization in drug delivery is silica nanostructures. In order to deliver an active agent as effective as possible, the silica nanostructures should be loaded with drug at an efficient speed and at a high capacity.

Silica nanostructures have a delicate structure. Therefore, the loading of the silica nanostructures must be controlled so integrity is not disrupted. Current loading methods include capillary action, in which silica nanostructures are combined with a solution containing active agent to be loaded, thereby forming “capillary loaded nanostructures”. However, this method results in a low load of active agent due to gas being entrapped in the nanostructures.

There exists a continuing need in the art to provide more efficient methods of loading active agents into silica nanostructures.

All publications referred to herein are hereby incorporated by reference in their entireties for all purposes.

### SUMMARY OF THE INVENTION

It is an object of the present invention to provide a method of loading silica nanostructures with active agent.

It is a further object of the present invention to provide silica nanostructures with an increased load of active agent.

The above objects of the invention, and others, can be obtained by the present invention, which in certain embodiments is directed to a method of loading silica nanostructures with an active agent, the method comprising: (a) exposing a mixture comprising a liquid medium containing an active agent and silica nanostructures to reduced air pressure for a time period; and (b) returning the mixture to atmospheric air pressure at the end of the lime period.

The time period of reduced pressure can be any suitable time to achieve loading of the nanostructures, e.g., from 1 second to 10 minutes or more. In certain embodiments, the time period can be, e.g., less than 90 seconds, less than 2 minutes, less than 3 minutes or less than 5 minutes. In other embodiments, the lime period can be. e.g., from about 15 seconds to about 5 minutes, from about 30 seconds to about 3 minutes, or from about 1 minute to about 2 minutes. The overall time will depend on factors such as the amount of pressure and the amount of nanotubes being loaded with active agent.

By carrying out the steps of the present invention, trapped air is removed from the nanostructures, allowing for the liquid medium to replace the trapped air aid fill the evacuated areas.

In one embodiment, a portion of the air escapes from the nanostructures during the time period in which the air pressure is reduced. During the time period in which the air pressure is reduced, an, trapped air in the nanostructures tends to expand and take up more volume as compared to the original amount of air under atmospheric pressure. Upon return to atmospheric pressure, the volume of the remaining air shrinks and will be displaced by the liquid medium, thereby filling the nanotubes with active agent.

In alternative embodiments, all or substantially all of the trapped air in the nanostructures is removed during the time of reduced air pressure and is displaced by the liquid medium prior to return to atmospheric pressure.

In another embodiment the nanotubes are filled with the liquid medium both during the time of reduced air pressure and upon return to atmospheric pressure.

In preferred embodiments, during the time of reduced air pressure, there is substantially no separation of the liquid medium from the silica nanostructures.

The method of the present invention provides an improvement in that the amount of liquid medium (and thus active agent) loaded into silica nanostructures is thereby increased, as compared to loading with the same conditions but without reduced air pressure (e.g., during capillary action). In certain non-limiting embodiments, the method of the present invention can be used to prepare nanostructures that are filled to a capacitor of greater than about 75%, greater than about 85%, greater than about 95%, or greater than about 99%.

In a further embodiment, the method of the present invention further comprises the step of applying increased air pressure to the mixture to further facilitate or increase the loading of the nanostructures. This step of increasing air pressure may be conducted in the time period before and/or after the time period of reduced air pressure.

In certain embodiments, the method further comprises the additional step of isolating the resulting active agent-loaded nanostructures from the liquid medium. In certain embodiments, the isolation is performed by a method selected from filtration, lyophilization, spray drying, evaporation, or a combination thereof. In certain embodiments, the isolated active agent-loaded nanostructures are in the form of particles.

In further embodiments, the present invention is directed to a drug delivery formulation comprising silica nanostructures loaded with a therapeutically active agent to a capacity of greater than 75%.

The term “capacity” when used in the context of “nanostructures loaded with active agent at a capacity greater than X %,” and the like, means that the inner volume of the nanostructures is filled with active agent above the specified percentage.

The term “BET surface area” refers to the rule for the physical adsorption of gas molecules on a solid surface, which is the basis for conducting an analysis technique for measuring the specific surface area of a material. See S. Brunauer, P. H. Emmett and E. Teller, J. Am. Chem. Soc., 1938, 60, 309, hereby incorporated by reference.

The term “air” is not limited to atmospheric air, but is intended to encompass any gas or mixture of gases.

“TMOS fibers” are nanostructures prepared utilizing tetramethyl orthosilicates.

“SBA-15” (Santa Barbara amorphous) are mesoporous silica nanostructures consisting of long-range channels. See, e.g., Zhao et al., Triblock Copolymer Syntheses of Mesoporous Silica with Periodic 50 to 300 Angstrom Pores, Science Vol. 279, 548-552 (1998).

The term “simulated body fluid” means an acellular simulated body fluid that has inorganic ion concentrations similar to those of human extracellular fluid, in order to reproduce formation of apatite on bioactive materials in vitro. Simulated body fluid is often referred to as Kokubo solution (see, e.g., Kokubo et al. in J. Biomed. Mater. Res. 24 (1990).

In certain embodiments, the simulated body fluid can have the approximate ion concentrations set forth in Table I below:

TABLE I
Ion concentrations of the simulated
body fluid and human blood plasma.

Concentration (mmol/dm³)

| Ion | Simulated body fluid (SBF) | Human blood plasma |
|---|---|---|
| Na⁺ | 142.0 | 142.0 |
| K⁺ | 5.0 | 5.0 |
| Mg²⁺ | 1.5 | 1.5 |
| Ca²⁺ | 2.5 | 2.5 |
| Cl⁻ | 147.8 | 103.0 |
| HCO₃⁻ | 4.2 | 27.0 |
| HPO₄²⁻ | 1.0 | 1.0 |
| SO₄²⁻ | 0.5 | 0.5 |

In certain embodiments of the present invention, the nanostructures are in a form such as tubes, rods, cylinders or mesostructures (e.g., structures with 2-5 nm pores). The nanostructures can have any morphology, such as spheres, fibers, discoids, origami (e.g., having a folded appearance) and mixtures thereof. However, the nanostructures can have any symmetrical or unsymmetrical form. When the nanotubes are aggregated, the space in between the nanostructures is also considered to constitute part of the “inner volume,” and can be filled with active agent along with the inner volume present in each of the individual nanostructures.

### BRIEF DESCRIPTION OF THE DRAWINGS

FIG. 1 shows a generalized diagram for synthesizing silica nanotubes.

FIG. 2 is a graph which compares discoids loaded with lidocaine by conventional methods versus discoids loaded by a method of the present invention.

### DETAILED DESCRIPTION

The present invention is generally directed to methods for increasing the amount of active agent that can be loaded into silica nanostructures. In one embodiment, the method comprises the steps of: (a) exposing a mixture comprising a liquid medium having an active agent and silica nanostructures to reduced air pressure for a time period; and (b) returning the mixture to atmospheric air pressure at the end of the time period. These steps can be repeated one or more times. Preferably, the method results in the silica nanostructures being loaded with the active agent at a capacity greater than 75%. When applying reduced air pressure to the mixture, the active agent is preferably dissolved or suspended in the liquid medium.

The extent of reduced air pressure applied to the mixture can be any suitable amount that results in an efficient and effective loading of active agent. In certain embodiments, the invention also includes subjecting the mixture to increased air pressure for a further time period in order to “force” the liquid medium (with active agent) into the nanostructures. Such time period of increased air pressure can be before and/or after the time period of reduced air pressure. In certain embodiments, the reduced air pressure is less than 100 kPa, or from about 10 kPa to about 90 kPa, or from about 20 kPa to about 80 kPa, or from about 30 kPa to about 70 kPa, or from about 40 kPa to about 60 kPa.

Silica nanostructures can be loaded with the active agent according to the present invention in an amount of about 0.5% active agent w/w or greater, or about 1% active agent w/w or greater, or about 5% active agent w/w or greater. However, a benefit of the present invention is that it can provide silica nanostructures having a high load of active agent. In certain embodiments, the silica nanostructures can be loaded with the active agent to a capacity of greater than about 75%, greater than about 85%, greater than about 95%, greater than about 98%, or greater than about 99%.

The high load silica nanostructures of the present invention can be incorporated into a pharmaceutical formulation for drug delivery.

In certain embodiments the high load silica nanostructures of the present invention when exposed to simulated body fluid provide at least one of the following release parameters: greater than 40 μgs of active agent released per mg of nanostructure after 0.5 hour, greater than 45 μgs of active agent released per mg of nanostructure after 1 hour, greater than 48 μgs of active agent released per mg of nanostructure after 2 hours, greater than 50 μgs of active agent released per mg of nanostructure after 3 hours, greater than 52 μgs of active agent released per mg of nanostructure after 5 hours, or greater than 55 μgs of active agent released per mg of nanostructure after 8 hours.

In further embodiments, the high load silica nanostructures of the present invention when exposed to simulated body fluid provide at least one of the following release profiles: greater than 42 μgs of active agent released per mg of nanostructure after 0.5 hour, greater than 47 μgs of active agent released per mg of nanostructure after 1 hour, greater than 52 μgs of active agent released per mg of nanostructure after 2 hours, greater than 55 μgs of active agent released per mg of nanostructure after 3 hours, greater than 58 μgs of active agent released per mg of nanostructure after 5 hours, or greater than 60 μgs of active agent released per mg of nanostructure after 8 hours.

In certain embodiments, the high load silica nanostructures of the present invention when exposed to simulated body fluid provide at least one of the following release parameters: from about 40 μgs to about 55 μgs of active agent released per mg of nanostructure after 0.5 hour, from about 45 μgs to about 60 μgs of active agent released per mg of nanostructure after 1 hour, from about 48 μgs to about 65 μgs of active agent released per mg of nanostructure after 2 hours, from about 50 μgs to about 70 μgs of active agent released per mg of nanostructure after 3 hours, from about 52 μgs to about 75 μgs of active agent released per mg of nanostructure after 5 hours, or from about 55 μgs to about 55 μgs of active agent released per mg of nanostructure after 80 hours.

In further embodiments, the high load silica nanostructures of the present invention exhibit at least 10%, 20% or 30% greater loading of active agent as compared to silica nanostructures identically prepared and loaded but without exposure to reduced air pressure according to the present invention.

In further embodiments, the high load silica nanostructures of the present invention exhibit at least 10%, 20% or 30% greater release of active agent as compared to silica nanostructures identically prepared and loaded but without exposure to reduced air pressure according to the present invention (e.g., when exposed to simulated body fluid at 0.5 hour, 1 hour, 2 hours, 3 hours, 5 hours or 8 hours).

In further embodiments, the high load silica nanostructures of the present invention exhibit at least 10% to 50% greater release of active agent as compared to silica nanostructures identically prepared and loaded but without exposure to reduced air pressure according to the present invention (e.g., when exposed to simulated body fluid at 0.5 hour, 1 hour, 2 hours, 3 hours, 5 hours or 8 hours).

In certain embodiments, the silica nanostructures of the present invention comprise nanotubes. The nanotubes can be closed on one end, closed on both ends, or open at both ends. In an alternative embodiment, the nanotubes can be a mixture of nanotubes that are closed on one end, open at both ends, and closed at both ends. For nanotubes that are closed on both ends, the active agent can be filled by permeating through imperfections in the body of the nanotube.

The silica nanostructures of the present invention can have a mean particle size selected from the group consisting of less than about 1 micron, less than about 800 nm, less than about 700 nm, less than about 600 nm, less than about 500 nm, less than about 400 nm, less than about 300 nm, less than about 290 nm, less than about 280 nm, less than about 270 nm less than about 260 nm, less than about 250 nm, less than about 240 nm, less than about 230 nm, less than about 220 nm, less than about 210 nm, less than about 200 nm, less than about 190 nm, less than about 180 nm, less than about 1770 nm, less than about 160 nm, less than about 1550 nm, less than about 140 nm, less than about 1330 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm

In certain embodiments, the silica nanostructures of the present invention have a mean diameter of from about 0.1 nm to about 20 nm, from about 1 nm to about 10 nm, or from about 2.5 nm to about 7.5 nm.

In other embodiments, the nanostructures of the present invention have a mean BET surface area from about 100 m²/g to about 5000 m²/g, from about 250 m²/g to about 2500 m²/g, or from about 500 m²/g to about 2000 m²/g.

In other embodiments, the nanostructures of the present invention have a mean volume of about 0.01 cc/g to about 5 cc/g, from about of 0.05 cc/g to about 3 cc/g, or from about 0.1 cc/g to about 2 cc/g.

In embodiments where the nanostructures are nanotubes, the nanotubes can have a morphology such as spheres, discoids, fibers, origami and a combination thereof.

The active agent utilized in the present invention can be, e.g., a small molecule or a peptide. Further, the active agent can either be hydrophilic or hydrophobic.

The term “active agent”, as used herein, may be selected from systemically active therapeutic agents, locally active therapeutic agents, disinfecting agents, chemical impregnants, cleansing agents, deodorants, fragrances, dyes, animal repellents, insect repellents, fertilizing agents, pesticides, herbicides, fungicides, plant growth stimulants, and the like. Active agents may be hydrophilic or hydrophobic.

The term “therapeutic agent”, as used herein, includes biological agents. e.g., small molecules, polypeptides, antihistamines (e.g., dimenhydrinate, diphenhydramine, chlorpheniramine and dexchlorpheniramine maleate), opioid analgesics (e.g. alfentanil, allylprodine, alphaprodine, anileridine, benz morphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl buty rate, dipipanone, eptazocine, ethoheptazine, ethylmethylthiambutene, ethylmorphine, etonitazene fentanyl, heroin, hydrocodone, hydromorphone, hydroxy pethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, and tramadol), anesthetics (e.g., benzocaine, amethocaine, amylocaine, butacaine, butoxycaine, butyl aminobenzoate, chloroprocaine, chlormecaine, cyclomethycaine, isobutamben, meprylcaine, novacaine, oxybuprocaine, procaine, propipocaine, proxymetacine, tricaine, lidocaine, bupivacaine, butanilicaine, carticaine, cinchocaine, clibucaine, etidocaine, mepivacaine, oxethazaine, prilocaine, ropivacaine, ethyl p-piperidinoacetyl-aminobenzoate, tolycaine, trimecaine and vadocaine), chemotherapeutic agents, non-opioid analgesics such as para-aminophenol derivatives (e.g., acetaminophen and phenacetinsteroidal) and non-steroidal anti inflammatory agents (NSAIDS; such as aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam and isoxicam), anti-emetics (e.g., metoclopramide), anti-epileptics (e.g., phenytoin, meprobamate and nitrezepam), vasodilators (e.g., nifedipine, papaverine, diltiazem and nicardirine), anti-tussive agents and expectorants (e.g., codeine phosphate), anti-asthmatics (e.g. theophylline), antacids, anti-spasmodics (e.g. atropine, scopolamine), antidiabetics (e.g., insulin), diuretics (e.g., ethacrynic acid, bendrofluazide), anti-hypotensives (e.g. propranolol, clonidine), antihypertensives (e.g., clonidine, methyldopa), bronchodilators (e.g., albuterol), steroids (e.g., hydrocortisone, triamcinolone, prednisone), antibiotics (including penicillin antibiotics (e.g., amoxicillin, ampicillin, azlocillin, carbenicillin, cloxacillin, dicloxacillin, flucloxacillin, mezlocillin, nafcillin, penicillin, piperacillin, and ticarcillin), aminoglylcoside antibiotics (e.g., amikacin, gentamicin, kanamycin, neomycin, netilmicin, streptomycin, and tobramycin), cephalosporin antibiotics (e.g., cefadroxil, cefazolin, cephalexin, cefaclor, cefamandole, cefotetan, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, and cefepime), macrolide antibiotics (e.g., azithromycin, clarithromycin, dirithromycin, erythromycin, and troleandomycin), quinolone antibiotics (e.g., ciprofloxacin, enoxacin, gatifloxacin, levofloxacin, lomefloxacin, moxifloxacin, norfloxacin, ofloxacin, and trovafloxacin), sulfonamide antibiotics (e.g., mafenide, sulfacetamide, sulfamethizole, sulfasalazine, sulfisoxazole, and trimethoprim), tetracyclin antibiotics (e.g. demeclocycline, doxycycline, minocycline, oxytetracycline, and tetracycline), glycopeptide antibiotics (teicoplanin and vancomycin), and polypeptide antibiotics (e.g., bacitracin, colistin, and polymyxin B) as well as chloramphenicol, clindamycin, ethambutol, fosfomycin, furazolidone, isoniazid, linezolid, metronidazole, nitrofurantoin, pyrazinamide, rifampin, and spectinomycin), antihemorrhoidals, hypnotics, psychotropics, antidiarrheals, mucolytics, sedatives, decongestants, laxatives, vitamins, stimulants (including appetite suppressants such as phenylpropanolamine), as well as salts, hydrates, and solvates of the same. The above list is not meant to be exhaustive.

In other embodiments, the therapeutic agent is selected from one of the following drugs, or belongs to one of the following classes: platinum compounds (e.g. spiroplatin, cisplatin, and carboplatin), methotrexate, fluorouracil, adriamycin, mitomycin, ansamitocin, bleomycin, cytosine arabinoside, arabinosyl adenine, mercaptopolylysine, vincristine, busulfan, chlorambucil, melphalan (e.g., PAM, L-PAM or phenylalanine mustard), mercaptopurine, mitotane, procarbazine hydrochloride dactinomycin (actinomycin D), daunorubicin hydrochloride, doxorubicin hydrochloride, paclitaxel and other taxenes, rapamycin, manumycin A, TNP-470, plicamycin (mithramycin), aminoglutethimide, estramustine phosphate sodium, flutamide, leuprolide acetate, megestrol acetate, tamoxifen citrate, testolactone, trilostane, arnsacrine (m-AMSA), asparaginase (L-asparaginase) Erwina asparaginase, interferon α-2a, interferon α-2b, teniposide (VM-26), vinblastine sulfate (VLB), vincristine sulfate, bleomycin sulfate, hydroxyurea, procarbazine, and dacarbazine; mitotic inhibitors, e.g. etoposide, coichicine, and the vinca alkaloids, radiopharmaceuticals, e.g. radioactive iodine and phosphorus products; hormones, e.g. progestins, estrogens and antiestrogens; anti-helmintics, antimalarials, and antituberculosis drugs; biologicals, e.g. immune serums, antitoxins and antivenoms; rabies prophylaxis products; bacterial vaccines; viral vaccines, respirator, products. e.g. xanthine derivatives theophylline and aminophylline; thyroid agents, e.g. iodine products and anti-thyroid agents; cardiovascular products including chelating agents and mercurial diuretics and cardiac glycosides; glucagon; blood products, e.g. parenteral iron, hemin, hematoporphyrins and their derivatives; biological response modifiers, e.g. muramyldipeptide, muramyltripeptide, microbial cell wall components, lymphokines (e.g., bacterial endotoxin, e.g. lipopolysaccharide, macrophage activation factor), sub-units of bacteria (such as Mycobacteria, Corynebacteria), the synthetic dipeptide N-acetyl-muramyl-L-alanyl-D-isoglutamine; anti-fungal agents, e.g. ketoconazole, nystatin, griseofulvin, flucytosine (5-fc), miconazole, amphotericin B, ricin, cyclosporins, and .beta.-lactam antibiotics (e.g., sulfazecin); hormones. e.g. growth hormone, melanocyte stimulating hormone, estradiol, beclomethasone dipropionate, betamethasone, betamethasone acetate and betamethasone sodium phosphate, vetamethasone disodium phosphate, vetamethasone sodium phosphate, cortisone acetate, dexamethasone, dexaniethasone acetate, dexaniethasone sodium phosphate, flunisolide, hydrocortisone, hydrocortisone acetate, hydrocortisone cypionate, hydrocortisone sodium phosphate, hydrocortisone sodium succinate, methylprednisolone, methyl prednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide, fludrocortisone acetate, oxytocin, vassopressin, and their derivatives; vitamins. e.g. cyanocobalamin neinoic acid, retinoids and derivatives, e.g. retinol palmitate, and .alpha.-tocopherol; peptides, e.g. manganese super oxide dismutase; enzymes, e.g. alkaline phosphatase; anti-allergic agents, e.g. amelexanox; anti-coagulation agents. e.g. phenprocoumon and heparin, circulator drugs, e.g. propranolol; metabolic potentiators, e.g. glutathione, antituberculars, e.g. para-aminosalicylic acid, isoniazid, capreomycin sulfate cycloserine, ethambutol hydrochloride ethionamide, pyrazinamide, rifampin, and streptomycin sulfate; antivirals, e.g. amantadine azidothymidine (AZT, DDI, Foscarnet, or Zidovudine), ribavirin and vidarabine monohydrate (adenine arabinoside, ara-A); antianginals, e.g. diltiazem, nifedipine, verapamil, erythritol tetranitrate, isosorbide dinitrate, nitroglycerin (glyceryl trinitrate) and pentaerythritol tetranitrate; anticoagulants, e.g. phenprocoumon, heparin; antibiotics, e.g. dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin including penicillin G and penicillin V, ticarcillin rifampin and tetracycline; antiinflammatories, e.g. diflunisal, ibuprofen, indomethacin, meclofenamate, mefenamic acid, naproxen, oxyphenbutazone, phenylbutazone, piroxicam, sulindac, tolmetin, aspirin and salicylates; anti protozoans, e.g. chloroquine, hydroxychloroquine, metronidazole, quinine and meglumine antimonate, antirheumatics, e.g. penicillamine, narcotics, e.g. paregoric; opiates, e.g. codeine, heroin, methadone, morphine and opium; cardiac glycosides, e.g. deslanoside, digitoxin, digoxin, digitalin and digitalis; neuromuscular blockers, e.g. atracurium mesylate, gallamine triethiodide, hexafluorenium bromide, metocurine iodide, pancuronium bromide, succinylcholine chloride (suxamethonium chloride), tubocurarine chloride and vecuronium bromide; sedatives (hypnotics), e.g. amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam and triazolam; local anesthetics, e.g. bupivacaine hydrochloride, chloroprocaine hydrochloride, etidocaine hydrochloride, lidocaine hydrochloride, mepivacaine hydrochloride, procaine hydrochloride and tetracaine hydrochloride; general anesthetics, e.g. droperidol, etomidate, fentanyl citrate with droperidol, ketamine hydrochloride, methohexital sodium and thiopental sodium; and radioactive particles or ions, e.g. strontium, iodide rhenium and yttrium.

In certain embodiments, the therapeutic agent is an analgesic selected from the group consisting of codeine, hydromorphone, hydrocodone, oxycodone, dihydrocodeine, dihy dromorphine, morphine, tramadol and oxymorphone.

In certain embodiments, the therapeutic agent is an anesthetic selected from the group consisting of bupivacaine, chloroprocaine, etidocaine hydrochloride, lidocaine, mepivacaine, novacaine, procaine and tetracaine.

The recitation of any active agent herein is meant to include any salt, hydrate or solvate thereof of the base compound.

Nanostructures are microscopic particles synthesized to a pre-determined range of microscopic sizes and shapes, depending on the method of synthesis. For example, nanostructures may be cylindrical, spherical or hexagonal, open-ended, or closed-ended, and have a range of diameters and lengths. Variations in the starting materials, surfactants used, concentrations, temperature and pH can all play a role in determining the type and physical attributes. e.g., morphology, of the nanostructure synthesized. Current methods of nanostructure synthesis have been discussed by Kievsky et al., IEEE Trans. on Nanotech., 4:5, the disclosure of which is hereby incorporated by reference in its entirety. Also see Naik et al., Morphology Control of Mesoporous Silica Particles, J. Phys. Chem. C 2007, 111, 11168-11173.

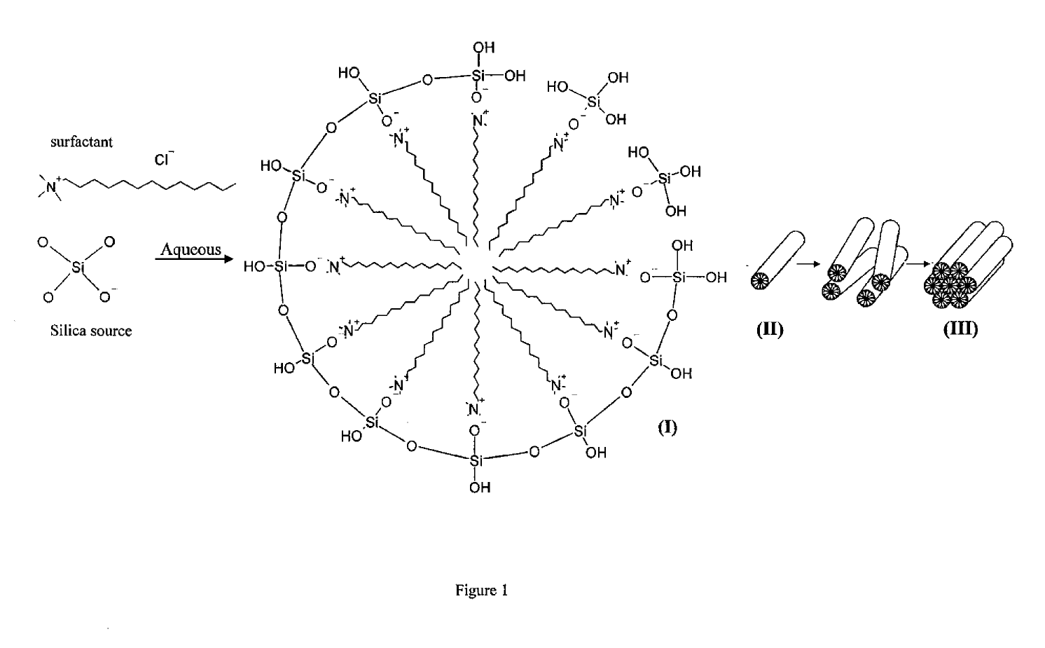

A generalized example of the synthesis of silica nanotubes is depicted in FIG. 1, and described in more detail below. Preferably, the silica source used is tetramethyl orthosilicates (TMOS) or tetraethyl orthosilicate (TEOS); however those skilled in the art will be aware of other silica sources that may be used in the present invention.

Silica nanotubes can also be prepared using, e.g., sol-gel template synthesis as described in Lakshmi, B. B., Patrissi, C. J., Martin, C. R., “Sol-Gel Template Synthesis of Semiconductor Oxide Micro- and Nanostructures.” Chem. Mater., 1997, 9, 2544 2550; Lakshmi, B. B., Dorhout, P. K., Martin. C. R., “Sol-Gel Template Synthesis of Semiconductor Nanostructures,” Chem. Mater., 1997, 9, 857, 862. The contents of these publications are hereby incorporated by reference in their entireties. Accordingly the template membrane is immersed into a standard tetraethyl orthosilicate or tetramethyl orthosilicate solution so that the solution fills the pores. After the desired immersion time, the membrane is removed, air dried and then cured at 150° C. This process yields silica nanotubes lining the pore Stalls of the membrane plus silica surface films on both faces of the membrane. The surface films are removed by briefly polishing with slurry of alumina particles. The nanotubes are then liberated by dissolving the template membrane, and collected by filtration.

The outside diameter of the nanotube can be controlled by varying the pore diameter of the template membrane; the length of the nanotube can be controlled by varying the thickness of the template membranes, and the inside diameter of the nanotube can be controlled by varying the immersion time in the solution. The template membrane pore diameter can be varied to produce nanotubes having diameters, e.g., from 5 nm to 100 μm. Likewise, the template membrane thickness can be varied to give nanotubes having a length, e.g., from 5 nm to 100 μm.

The preferred dimensions of nanostructures depend on the intended application. Because of concerns regarding occlusion of blood flow at the microvasculature level, it is important not to make nanostructures too large for intravenous applications. For comparison purposes, a red blood cell is about 7000 nm in diameter, which corresponds to the diameter of the smallest capillaries in the human body. If the nanostructure has suitable surface property characteristics (surface charge and hydrophilicity/hydrophobicity balance) and has a dimension typically less than 10 nm, it may be directly excreted from the body by the kidneys. Alternatively, if the nanostructure is larger and biodegradable, it will not be directly eliminated from the body, but rather will be degraded into its smaller components, which in turn will be eliminated.

When the nanostructure is intended for in-vivo use, and particularly when it is to be injected into the blood stream, the maximum dimension (e.g., length of a nanotube) is preferably less than 500 nm. The “maximum dimension” of the nanostructure is the maximum distance between two points of the nanostructure.

Nanostructures intended for in-vivo use should preferably have a diameter of less than 200 nm. In one embodiment, nanotubes for in-vivo use have a maximum dimension of 100 nm or less.

The high load silica nanostructures of the present invention may be administered by a variety, of routes, for example, by enteral, oral, buccal, rectal, vaginal, dermal, nasal, bronchial, tracheal, pulmonary, parenteral, subcutaneous, intravenous, intramuscular, intraocular, or intraperitoneal route.

A preferred formulation of high load silica nanostructures of the present invention includes controlled release formulations that permit deliver) of the drug over an extended period of time. In certain embodiments, the high load silica nanostructures of the present invention will provide the desired controlled release, without the necessity for including further controlled release materials.

For oral administration, the high load silica nanostructures of the present invention can be formulated in a solid or liquid dosage form. The solid dosage form can be an oral solid dosage form such as a tablet or a capsule. A liquid dosage form can be a suspension or an emulsion.

Suitable excipients include fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropyl methylcellulose, sodium carboxy methylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, non-regulating agents and lubricants, for example, silica, talc, stearic acid or salts thereof, such as magnesium stearate or calcium stearate, and/or polyethylene glycol.

Liquid preparations may be prepared by suspending the high load silica nanostructures of the present invention in an aqueous or non-aqueous pharmaceutically acceptable medium which may also contain sweetening agents, flavoring agents, and preservative agents as are known in the art.

For parenteral administration, the high load silica nanostructures of the present invention man be suspended in a physiologically acceptable pharmaceutical carrier and administered as a suspension. For example, suitable suspensions of the nanostructures as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions may contain substances which increase the viscosity of the suspension, and include, for example, sodium carboxymethyl cellulose, sorbitol, and/or dextran. Optionally, the suspension may also contain stabilizers.

The high load silica nanostructures of the present invention can also be applied topically. This can be accomplished by preparing a suspension of the nanostructures using a solvent known to promote transdermal absorption such as ethanol or dimethyl sulfoxide (DMSO) with or without other excipients. Preferably, topical administration will be accomplished using a transdermal patch either of the reservoir and porous membrane type or of a solid matrix variety.

For administration into a surgical or wound site, the high load silica nanostructures of the present invention may be combined with a biodegradable or bioerodible carrier based on materials such as polylactides and poly-lactide-co-glycolides and collagen formulations. Such materials may be in the form of solid implants, sponges, and the like.

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXAMPLES

### Example 1

### Synthesis of Nanotubes

As generally presented in FIG. 1, under aqueous conditions, the hydrophobic tails of a surfactant, such as quaternized ammonium compounds, will tend to aggregate to form micelles under appropriate conditions. These micelles are then sequestered by the negatively charged silica source. This spatial arrangement allows for orderly condensation of neighboring silicates. Further micelle aggregation leads to the formation of capillaries, which then undergo self-association. Surfactant may be removed from the capillaries via heat, preferably at about 500° C. (calcination).

### Example 2

### Lidocaine Filled-Nanotubes Via Vacuum Loading

Equipment: 1.69 mg of nanostructures (in the form of TMOS-fibers) (weight in 1.8 ml cryotubes)

Small lypholization container to hold the 1.8 ml cryo tube for vacuum loading

Small oilless vacuum pump (typically pull 10-15 torr.)

Spin-X centrifuge tube filter 0.45 mM Nylon (to separate nanotubes from their loading solution; do the water washes (to remove excess lidocaine); and for air-drying)

400 ml of 25% wt aqueous lidocaine was degassed for about 2 to 3 minutes under vacuum. The degassed solution of lidocaine as added to the TMOS-fibers. A vacuum of approximately 4 torr was applied four times and released back to atmospheric pressure in between each cycle. Gas bubbles were observed emanating from the nanotubes at the bottom of the 1.8 ml cryo tube. The solution with the nanotubes was transferred to a spin-x-lc filter insert (Costar 8170) and filtered with 5 psi, N₂ gas. The loaded fibers were washed with H₂O three times (75 ul each wash) and air dried for at least 2 days.

The procedure was repeated using nanotubes of different morphologies using:

Discoids—1.77 mg

SBA-15—1.49 mg

### Example 3

### Lidocaine Filled-Nanotubes Via Vacuum Loading

The loading procedure was repeated as above with:

51% wt. lidocaine at pH 5.7 (50% wt HCl.1H₂O and 1% wt. free base) and 1.35 mg TMOS-fibers.

51% wt. lidocaine, pH 5.7 and 1.63 mg TMOS-fibers; and

51% wt lidocaine, pH 3.1 and 1.29 mg TMOS-fibers.

The next day, the fibers loaded with 51% lidocaine (at pH 5.7, and at pH 3.1) were washed, and then air dried for several days.

Initial release studies were conducted for each nanotube morphology discussed above. Each nanotube type was separately loaded with 1, 10 & 25 wt. % aqueous lidocaine HCl.

TMOS fibers with 25 wt % load gave the most extended release profile in simulated body fluid.

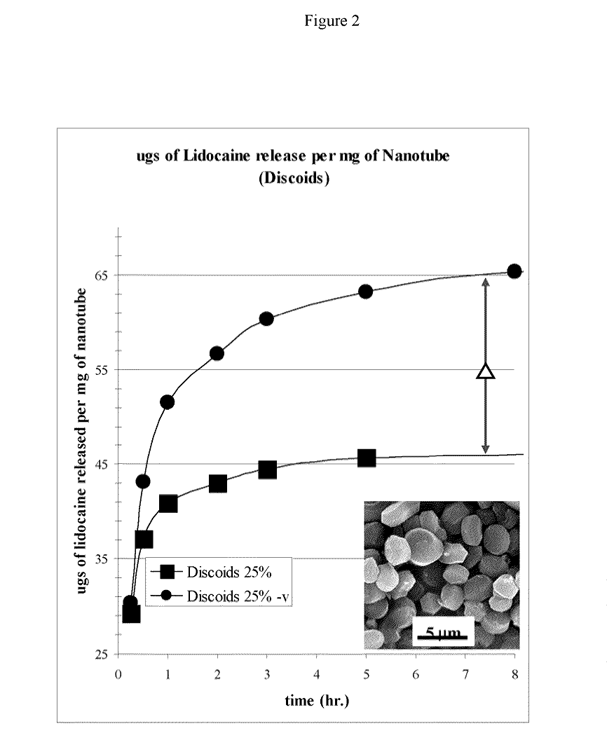

Vacuum loading had the largest positive loading differential on discoid nanotube types as compared to non-vacuum loading. The results are set forth in FIG. 2.

The present invention is not to be limited in scope by the specific embodiments disclosed in the examples which are intended as illustrations of a few aspects of the invention. Any embodiments that are functionally equivalent are within the scope of this invention. Indeed, various modifications of the invention in addition to those shown and described herein will become apparent to those skilled in the art and are intended to fall within the scope of the appended claims.

1. A method of loading silica nanostructures with an active agent comprising:
(a) exposing a mixture comprising a liquid medium containing an active agent and silica nanostructures to reduced air pressure for a time period; and
(b) returning the mixture to atmospheric air pressure at the end of the time period.

2-4. (canceled)

5. The method of claim 1, wherein there is substantially no separation of the liquid medium from the silica nanostructures during the time period of reduced air pressure.

6. The method of claim 1, wherein the nanostructures are loaded with the active agent at a capacity greater than 75%

7. The method of claim 1, further comprising the step of applying increased pressure to the mixture.

8. (canceled)

9. The method of claim 1, wherein the nanostructures are in a form selected from the group consisting of tubes, rods, cylinders, mesostructures and mixtures thereof.

10. The method of claim 1, wherein the nanostructures have a morphology selected from the group consisting of spheres, fibers, discoids, origami and mixtures thereof.

11. The method of claim 1, wherein the nanostructures comprise nanotubes.

12-14. (canceled)

15. The method of claim 11, wherein the nanotubes have a mean diameter of from about 0.1 mm to about 20 nm.

16-17. (canceled)

18. The method of claim 11, wherein the nanotubes have a mean BET surface area from about 100 m²/g to about 5000 m²/g.

19-20. (canceled)

21. The method of claim 11, wherein the nanotubes have a mean volume of 0.01 cc/g to about 5 cc/g.

22-24. (canceled)

25. The method of claim 1, wherein the active agent is a small molecule or a peptide.

26-27. (canceled)

28. The method of claim 1, wherein the nanostructures are loaded with the active agent at a capacity greater than 85%.

29-31. (canceled)

32. The method of claim 1, further comprising the step of isolating particles comprising the active agent-loaded nanostructures from the liquid medium.

33. (canceled)

34. The method of claim 32, wherein the particles comprise the active agent in an amount from about 0.5% to about 90% active agent (w/w).

35-36. (canceled)

37. The method of claim 1, wherein the active agent is selected from the group consisting of an analgesic or a local anesthetic.

38-39. (canceled)

40. A drug delivery formulation comprising silica nanostructures loaded with an active agent to a capacity of greater than 75%.

41. The formulation of claim 40, wherein the nanostructures are nanotubes.

42. The formulation of claim 40, wherein the nanotubes have a morphology selected from the group consisting of spheres, fibers, discoids, origami and mixtures thereof.

43. The formulation of claim 40, wherein the nanostructures are loaded with the active agent at a capacity greater than 85%.

44-46. (canceled)

47. The formulation of claim 40, wherein the active agent is selected from the group consisting of an analgesic or a local anesthetic.

48-49. (canceled)
